(19) United States
(12) Patent Application Publication
(10) Pub. No.: US 2026/0165960 A1
(43) Pub. Date: Jun. 18, 2026

(54) FOOD PRODUCT FOR MYOFUNCTIONAL PREVENTION OF OROFACIAL DISORDERS IN THE HUMAN ORAL CAVITY AND ITS USE

(71) Applicant: Borisova Kristina Vitalievna, Moscow (RU)

(72) Inventor: Borisova Kristina Vitalievna, Moscow (RU)

(21) Appl. No.: 19/423,197

(22) Filed: Dec. 17, 2025

(30) Foreign Application Priority Data
Dec. 17, 2024 (RU) 2024138023

(51) Int. Cl. A61K 9/00; A61K 47/02; A61K 47/10; A61K 47/12; A61K 47/26; A61K 47/38; A61K 47/46; A61P 1/02

(52) U.S. Cl. CPC A61K 9/0056; A61K 47/02; A61K 47/10; A61K 47/12; A61K 47/26; A61K 47/38; A61K 47/46; A61P 1/02

(57) ABSTRACT

A food product for the myofunctional prevention of orofacial disorders in the human oral cavity contains biocompatible materials: xylitol, agar-agar, carboxymethylcellulose, magnesium stearate, colloidal silicon dioxide, natural flavoring, and, optionally, natural coloring selected from beet coloring and dry turmeric extract. The content of components in one tablet in mg is as follows: xylitol 120.0, agar-agar 40.0, carboxymethylcellulose 23.0, caramel flavoring 6.0, xanthan gum 5.0, colloidal silicon dioxide 3.0, magnesium stearate 3.0 or xylitol 120.0, agar-agar 40.0, carboxymethylcellulose 20.0, forest berry flavoring 5.0, beetroot dye 4.0, xanthan gum 5.0, colloidal silicon dioxide 3.0, magnesium stearate 3.0 or xylitol 120.0, agar-agar 40.0, carboxymethylcellulose 23.0, mango flavoring 5.0, dry turmeric extract 1.0, xanthan gum 5.0, colloidal silicon dioxide 3.0, magnesium stearate 3.0. The use of this food product for myofunctional prevention of orofacial disorders in the human oral cavity is also proposed.

### DESCRIPTION OF THE INVENTION

The invention relates to the food industry, in particular to myofunctional prevention of orofacial disorders. The food product according to the present invention is intended for myofunctional prevention of orofacial disorders in the human oral cavity, as well as for the prevention of incorrect physiological nasal breathing and the prevention of caries and strengthening of tooth enamel, especially in consumers with orthodontic appliances.

### STATE OF THE ART

A known agent for the prevention and treatment of infectious and inflammatory diseases of the oropharynx, containing an active ingredient and excipients, characterized in that it contains beet pectin, edible agar, sodium carboxymethylcellulose, sugar, and calcium stearate, characterized in that it is intended for palatal or buccal administration and has self-fixing properties (patent RU 2216320).

There is a known muscle trainer for functional correction of the tongue position called “Myospots” (https://myospots.com, https://osobaya-territoria.com/tproduct/790094894-938660683201-miospot), containing seaweed agar, cellulose gum and xanthan gum, stevia, natural fruit flavoring (banana, blueberry, or strawberry), natural coloring, and potassium sorbate. This product was selected as the prototype for the present invention.

### FIELD OF THE INVENTION

The position, posture, and/or movement of the tongue are crucial in a person's daily life. The position of the tongue on the palate is important for the development of the upper jaw and prevents the movement of facial muscles during swallowing. In addition, excessive use of these muscles during swallowing or rest can contribute to malocclusion of the oral cavity. It is believed that deviation of the tongue from its desired position can lead to significant breathing problems. For example, mouth breathing plays a crucial role in asthma and can increase the risk of infections in the mouth and throat. In addition, mouth breathing can cause pollutants and germs to enter the lungs directly. In this regard, myofunctional disorder can arise from abnormal tongue position during rest, swallowing, and/or speech and lead to significant problems such as malocclusion, tooth displacement, dysphagia, snoring, apnea, hypersalivation, and speech problems, which in turn necessitates orthodontic treatment, speech therapy, and the use of various trainers for correct tongue position. Thus, myofunctional prevention of orofacial disorders is crucial for many people.

An important group of consumers are people with orthodontic appliances, for whom oral health is a top priority. Recently, the demand for orthodontic treatment has increased significantly. Orthodontic appliances in the oral cavity, if not properly cared for, can adversely affect the periodontium and hard tissues of the teeth. Therefore, special attention should be paid to preparing patients for long-term orthodontic treatment using effective means of preventing dental and oral mucosal diseases. Although the effectiveness of fixed orthodontic appliances is not in doubt, the percentage of complications identified during orthodontic treatment remains high. During treatment, the role of dental plaque microorganisms increases as the number of retention points increases, and the influence of protective factors such as self-cleaning of teeth and the remineralizing potential of saliva is significantly reduced. Orthodontic appliances in the oral cavity, if not properly cared for, can adversely affect the periodontium, hard tooth tissues, and the oral mucosa. Changes in the color of the tooth enamel around the braces, the formation of white spots and enamel defects, hyperesthesia of individual teeth after braces removal, gingivitis, and periodontitis are also possible. In this category of patients, who constitute one of the most likely risk groups, the condition of the periodontium and its response to orthodontic treatment require special attention, since morphofunctional disorders caused by anomalies of the dentofacial system are themselves powerful pathogenetic factors that cause the initiation and development of periodontal diseases. Even in adolescents with orthognathic occlusion, there is an extremely high incidence of chronic catarrhal gingivitis (Karakov K. G. et al.: Evaluation of the effectiveness of oral hygiene in patients undergoing orthodontic treatment//Modern problems of science and education.—2016.—No. 5). To prevent such disorders, a food product for myofunctional prevention of orofacial disorders in the human oral cavity has been developed in accordance with the present invention (“Foxyspot”), which has properties that allow it to be effectively used for the prevention of the above-mentioned problems by people with orthodontic disorders and orthodontic appliances.

The use of a food product for myofunctional prevention of orofacial disorders in the human oral cavity according to the present invention (“Foxyspot”) due to its carefully selected composition allows the following technical results to be achieved:
Improved prevention of recurrence for consumers who have undergone orthodontic treatment;
Increased effectiveness of caries prevention;
Improved prevention of oral and throat infections;
No irritation of the oral mucosa;
Improved quality of life for consumers;
Improvement (enhancement) of the organoleptic properties of food products

Expansion of the range (arsenal) of technical means for myofunctional prevention of orofacial disorders.

### SUMMARY OF THE INVENTION

The FoxySpot myofunctional correction food product is a tablet—an adhesive disc made of biocompatible materials—that is placed on the human palate. Foxyspot acts as a tactile reminder, a kind of kinesthetic marker, stimulating the tongue to maintain a physiologically correct position and stimulating the correct somatic type of swallowing. The formation of the correct palatal position of the tongue and somatic type of swallowing (correction of infantile type of swallowing), achieved by the absorption of the disc stuck to the palate, is especially important for consumers with orthodontic appliances.

A feature of the food product according to the present invention (Foxyspot) is the use of xylitol as a sweetener. Thanks to its carefully selected composition, the food composition according to the present invention has the following properties:

Reduces plaque formation and helps prevent tooth decay.

Strengthens tooth enamel through remineralization.

Prevents the growth of pathogenic bacteria such as Streptococcus mutans.

The FoxySpot food product according to the present invention has the following characteristics:

Secure attachment to the mucous membrane of the palate in 10 seconds without discomfort to the user.

Gradual dissolution within 20 to 60 minutes, eliminating the need to remove the disc.

Biocompatibility and safety for health, confirmed by clinical studies.

Foxyspot materials provide a soft texture and no irritation to the mucous membrane, in particular due to the xylitol content.

### Advantages of the Invention

### Ease of Use.

Versatility: can be used in conjunction with orthodontic appliances and other therapeutic devices.

Environmental safety thanks to the use of natural ingredients.

Additional preventive benefits due to the inclusion of xylitol, in particular for the health of teeth and gums.

In some embodiments, the food product according to the present invention is a food product for myofunctional prevention of orofacial disorders in the human oral cavity, characterized by biocompatible materials that ensure adhesion to the mucous membrane of the palate and gradual dissolution in the oral cavity, distinguished by the fact that it contains xylitol as one of the biocompatible materials.

In some embodiments, the food product according to the present invention is a food product for myofunctional prevention of orofacial disorders in the human oral cavity, characterized by biocompatible materials that ensure adhesion to the mucous membrane of the palate and gradual dissolution in the oral cavity, where the biocompatible materials are xylitol, agar, carboxymethylcellulose, magnesium stearate, and colloidal silicon dioxide.

In some embodiments, the food product according to the present invention is a food product for myofunctional prevention of orofacial disorders in the human oral cavity, characterized by biocompatible materials that ensure adhesion to the mucous membrane of the palate and gradual dissolution in the oral cavity, where the biocompatible materials are xylitol, agar-agar, carboxymethylcellulose, magnesium stearate, colloidal silicon dioxide, natural flavoring, and, optionally, natural coloring.

In some embodiments, the food product according to the present invention is a food product for myofunctional prevention of orofacial disorders in the human oral cavity, characterized by biocompatible materials that ensure adhesion to the mucous membrane of the palate and gradual dissolution in the oral cavity.

In some embodiments, the food product according to the present invention is a food product for the myofunctional prevention of orofacial disorders in the human oral cavity, characterized by biocompatible materials that ensure adhesion to the mucous membrane of the palate and gradual dissolution in the oral cavity, where the biocompatible materials are xylitol, agar-agar, carboxymethylcellulose, magnesium stearate, colloidal silicon dioxide, and flavoring, and, optionally, natural coloring.

In some embodiments, the food product according to the present invention is a food product for myofunctional prevention of orofacial disorders in the human oral cavity, in which the natural flavoring is selected from mango flavoring, forest berry flavoring, and caramel flavoring.

In some embodiments, the food product according to the present invention is a food product for myofunctional prevention of orofacial disorders in the human oral cavity, in which the natural colorant is selected from beetroot colorant and dry turmeric extract.

In some embodiments, the food product according to the present invention is a disc with a diameter of 5 to 8 mm and a thickness of 3 mm.

In some embodiments, the food product according to the present invention is a food product for myofunctional prevention of orofacial disorders in the human oral cavity, characterized by the following component content:

Content in one tablet, mg

| | | Forest | |
|---|---|---|---|
| | Caramel | berry | Mango |
| Component | flavor | flavor | flavor |
| Xylitol | 120.0 | 120.0 | 120 |
| Agar-agar | 40 | 40 | 40 |
| Carboxymethylcellulose | 23.0 | 20.0 | 23.0 |
| Caramel flavoring | 6.0 | — | — |
| Xanthan gum | 5.0 | 5.0 | 5.0 |
| Forest berry flavoring | — | 5.0 | — |
| Mango Flavoring | — | — | 5.0 |
| Beetroot coloring | — | 4.0 | — |
| Colloidal silicon dioxide | 3.0 | 3.0 | 3 |
| Magnesium stearate | 3.0 | 3.0 | 3 |
| Turmeric extract, dry, n/s | — | — | 1 |
| Total | 200.0 | 200.0 | 200.0 |

In some embodiments, the food product according to the present invention is a food product for myofunctional prevention of orofacial disorders in the human oral cavity, characterized by a dissolution time in the oral cavity of 20-60 minutes.

In some embodiments, the food product according to the present invention is a food product for myofunctional prevention of orofacial disorders in the human oral cavity and adhesion to the human palate, characterized by biocompatible materials that ensure adhesion to the mucous membrane of the palate and gradual dissolution in the oral cavity, distinguished by the fact that it contains xylitol as one of the biocompatible components.

In some embodiments, the food product according to the present invention is a food product for myofunctional prevention of orofacial disorders in the human oral cavity and adhesion to the human palate, characterized by biocompatible materials that ensure adhesion to the mucous membrane of the palate and gradual dissolution in the oral cavity, where the biocompatible materials are xylitol, agar-agar, carboxymethylcellulose, magnesium stearate, and colloidal silicon dioxide.

In some embodiments, the food product according to the present invention is a food product for myofunctional prevention of orofacial disorders in the human oral cavity and adhesion to the human palate, characterized by biocompatible materials that ensure adhesion to the mucous membrane of the palate and gradual dissolution in the oral cavity, where the biocompatible materials are xylitol, agar-agar, carboxymethylcellulose, magnesium stearate, colloidal silicon dioxide, natural flavoring, and, optionally, natural coloring.

In some embodiments, the food product according to the present invention is a food product for myofunctional prevention of orofacial disorders in the human oral cavity and adhesion to the human palate, characterized by biocompatible materials that ensure adhesion to the mucous membrane of the palate and gradual dissolution in the oral cavity.

In some embodiments, the food product according to the present invention is a food product for myofunctional prevention of orofacial disorders in the human oral cavity and adhesion to the human palate, characterized by biocompatible materials that ensure adhesion to the mucous membrane of the palate and gradual dissolution in the oral cavity, where the biocompatible materials are xylitol, agar-agar, carboxymethylcellulose, magnesium stearate, colloidal silicon dioxide, and natural flavoring, and, optionally, natural coloring.

In some embodiments, the food product according to the present invention is a food product for myofunctional prevention of orofacial disorders in the human oral cavity and adhesion to the human palate, in which the natural flavoring is selected from mango-flavored flavoring, forest berry flavoring, and caramel flavoring.

In some embodiments, the food product according to the present invention is a food product for myofunctional prevention of orofacial disorders in the human oral cavity and sticking to the human palate, in which the natural colorant is selected from beetroot colorant and dry turmeric extract.

In some embodiments, the food product according to the present invention is a disc with a diameter of 5 to 8 mm and a thickness of 3 mm.

In some embodiments, the food product according to the present invention is a food product for myofunctional prevention of orofacial disorders in the human oral cavity and adhesion to the human palate, characterized by the following component content:

Content in one tablet, mg

| | | Forest | |
|---|---|---|---|
| | Caramel | berry | Mango |
| Component | flavor | flavor | flavor |
| Xylitol | 120.0 | 120 | 120 |
| Agar-agar | 40.0 | 40 | 40 |
| Carboxymethylcellulose | 23.0 | 20 | 23 |
| Caramel flavoring | 6.0 | — | — |
| Xanthan gum | 5.0 | 5.0 | 5.0 |
| Forest berries flavoring | — | 5.0 | — |
| Mango flavoring | — | — | 5.0 |
| Beetroot coloring | — | 4.0 | — |
| Colloidal silicon dioxide | 3.0 | 3.0 | 3.0 |
| Magnesium stearate | 3.0 | 3.0 | 3 |
| Turmeric extract, dry | — | — | 1 |
| plant extract n/s | | | |
| Total | 200.0 | 200.0 | 200.0 |

In some embodiments, the food product according to the present invention is a food product for myofunctional prevention of orofacial disorders in the human oral cavity and adhesion to the human palate, characterized by a dissolution time in the oral cavity of 20-60 minutes.

### EXAMPLES

The examples presented in this description of the invention do not limit the invention in any way.

### Example 1

This example provides examples of compositions of the food product “Foxyspot Tongue Muscle Trainer” that could not be obtained in the form of a tablet—an adhesive disc. The tablet either disintegrated or resulted in an adhesive “drop” form. These food products are provided as examples and are not limited to the data below.

TABLE 1
Composition of a mango-flavored tongue muscle
trainer using xylitol as a sweetener.

| Component | Content in one tablet, mg |
|---|---|
| Xanthan gum | 5.0 |
| Agar-agar | 41 |
| Xylitol | 124.0 |
| Carboxymethylcellulose | 24.0 |
| Colloidal silicon dioxide | 3 |
| Magnesium stearate | 3 |
| Mango flavoring | 6.0 |
| Total | 206.0 |

TABLE 2
Composition of caramel-flavored tongue muscle
trainer using xylitol as a sweetener.

| Component | Content per tablet, mg |
|---|---|
| Xanthan gum | 5 |
| Agar-agar | 41 |
| Xylitol | 124.0 |
| Carboxymethylcellulose | 24.0 |
| Colloidal silicon dioxide | 3 |
| Magnesium stearate | 3 |
| Caramel flavoring | 6.0 |
| Total | 206.0 |

TABLE 3
Composition of a forest berry-flavored tongue muscle
training device using xylitol as a sweetener.

| Component | Content per tablet, mg |
|---|---|
| Xanthan gum | 5.0 |
| Agar-agar | 41 |
| Xylitol | 124.0 |
| Carboxymethylcellulose | 24.0 |
| Colloidal silicon dioxide | 3 |
| Magnesium stearate | 3.0 |
| Forest berries flavoring | 6.0 |
| Total | 206.0 |

TABLE 4
Composition of the mango-flavored tongue muscle trainer
using dextrose monohydrate as a sweetener.

| Component | Content per tablet, mg |
|---|---|
| Xanthan gum | 10 |
| Agar-agar | 10 |
| Mango flavoring | 1.0 |
| Dextrose monohydrate | 100 |
| Microcrystalline cellulose | 73 |
| Colloidal silicon dioxide | 3 |
| Magnesium stearate | 3.0 |
| Total | 200.0 |

TABLE 5
Composition of caramel-flavored tongue muscle trainer
using dextrose monohydrate as a sweetener.

| Component | Content per tablet, mg |
|---|---|
| Xanthan gum | 10 |
| Agar-agar | 10 |
| Caramel flavoring | 1.0 |
| Dextrose monohydrate | 100 |
| Microcrystalline cellulose | 73 |
| Colloidal silicon dioxide | 3 |
| Magnesium stearate | 3.0 |
| Total | 200.0 |

TABLE 6
Composition of the forest berry-flavored tongue muscle
trainer using dextrose monohydrate as a sweetener.

| Component | Content per tablet, mg |
|---|---|
| Xanthan gum | 10 |
| Agar-agar | 10.0 |
| Forest berries flavoring | 1.0 |
| Dextrose monohydrate | 100 |
| Microcrystalline cellulose | 73 |
| Colloidal silicon dioxide | 3 |
| Magnesium stearate | 3.0 |
| Total | 200.0 |

### Example 2

The preventive effect of the food product according to the present invention (FoxySpot tongue muscle trainer) and Myospots compositions on the occurrence of orofacial disorders in consumers at high risk of developing them due to incorrect tongue position was investigated. Over a period of 32 weeks, the groups using FoxySpot and Myospots did not experience orofacial disorders such as hypersalivation, snoring, and mouth breathing in 85% of consumers, while the control group that did not take FoxySpot or Myospots, showed the above-mentioned orofacial disorders.

### Example 3

The preventive effect of the food product according to the present invention (FoxySpot tongue muscle trainer) on the formation of caries was studied in vitro on 130 intact teeth removed for orthodontic reasons. The teeth were first washed in running water with a brush, cleaned of dental deposits, and the crown surface was polished with a brush. A suspension was prepared from the Myospots and FoxySpot compositions using methods known in the art and applied to the surface of the teeth, and the applied suspension was left for a period of 20 to 60 minutes, after which the suspension was washed off with running water. Each group of teeth consisted of 10 teeth—3 groups for Myospots each with a different flavor and 3 groups for compositions “Foxyspot” each with a different flavor, as well as 3 groups for “Foxyspot” compositions according to example 1, each with a different flavor, and 3 groups for tongue muscle trainers with dextrose monohydrate according to example 1, each with a different flavor. In the control group of 10 teeth, an aqueous solution was applied to them, from which a suspension for the Myospots and FoxySpot compositions was prepared, after which they were also left for 40 minutes and then rinsed with running water. After the above actions, a glucose suspension and a Streptococcus mutans suspension were applied to the teeth, after which they were left for 4 hours and then also rinsed with running water. The above actions were repeated twice a day for 32 weeks. It was found that Myospots had a 15% preventive effect on the development of caries, while FoxySpots had a 40% preventive effect. The compositions from example 1 showed the above-mentioned preventive effect by 30% for compositions with xylitol and by 20% for compositions with dextrose monohydrate.

### Example 4

Caries prevention: using FoxySpot for 8 weeks reduced the risk of caries formation by 40% in high-risk users, while using Myospots reduced it by only 15%.

### Example 5

In testing the improvement in the prevention of recurrence for consumers who had undergone orthodontic treatment, 70 people participated, divided into 7 groups of 10 people each. The consumers took the FoxySpot food product according to the present invention—each group took its own FoxySpot composition with a specific flavor, or the consumers took the prototype—Myospot compositions with different flavors for each group, while the control group took neither FoxySpot nor Myospot. Consumption of the aforementioned products among respondents continued for 4 years. During this time, only one case of recurrence was recorded for consumers who took FoxySpot (among all three groups, i.e., out of 30 people)—in the group that took the caramel-flavored compositions, and three cases of recurrence were recorded among consumers who took Myospot (among all 3 groups, i.e., 30 people)—2 cases for banana-flavored compounds and 1 case for blueberry-flavored compounds, while in the control group of 10 people, 3 cases of recurrence were recorded. Thus, FoxySpot formulations have higher rates of relapse prevention among respondents who have undergone orthodontic treatment than the prototype.

With regard to achieving the technical result of improving the prevention of oral and throat infections, the applicant cites the following Example 6 and further explains that the correct position of the tongue in the mouth contributes to the prevention of respiratory infections, as it improves blood supply to the mouth and throat and reduces the likelihood and frequency of “mouth breathing”, which causes dry mouth, chronic rhinitis, and other problems, such as an increased risk of developing various infectious and inflammatory diseases of the mouth and throat and the occurrence of such diseases in a chronic form.

### Example 6

The preventive effect of FoxySpot compositions on the occurrence of infectious and inflammatory diseases of the mouth and throat was studied in comparison with the prototype. The study involved 70 people—10 people in each group. Three groups were formed for the Myospot compositions, each with a different flavor, and three groups for the FoxySpot compositions, each with a different flavor, as well as one control group that did not take FoxySpot or Myospot. Consumers took FoxySpot and Myospot for 8 months, and then the frequency and occurrence of infectious inflammatory diseases of the mouth and throat were recorded. Consumers using FoxySpot and Myospot formulations showed a preventive effect on the occurrence of infectious and inflammatory diseases of the mouth and throat compared to the control group. Foxyspot formulations showed a 20% improvement in the prevention of infectious and inflammatory diseases of the mouth and throat compared to the prototype (Myospot formulations). In addition, some consumers (10%) who took Myospot reported irritation of the oral mucosa and dry mouth, while none of the consumers who took FoxySpot reported such side effects.

### Example 7

Number of respondents who rated the organoleptic properties of the food product compositions according to the present invention (FoxiSpot) and the prototype (Myospot). A total of 120 people participated in the study—20 people in each of the 6 groups, where 3 groups tried FoxySpot compositions according to the present invention, each with a different flavor, and 3 groups tried Myospot compositions, each with a different flavor.

TABLE 7
Results of testing the organoleptic properties of FoxySpot
compositions compared to Myospot compositions.

| | | FoxySpot compositions | | | | | |
|---|---|---|---|---|---|---|---|
| | | | Forest | | Myospot compositions (prototype) | | |
| Indicator | Respondents' | Caramel | berry | Mango | Banana | Strawberry | Blueberry |
| name | assessment | flavor | flavor | flavor | flavor | flavor | flavor |
| Overall taste | Good (main | 14 | 15 | 14 | 12 | 11 | 13 |
| | indicator) | | | | | | |
| | Satisfactory | 4 | 3 | 3 | 4 | 5 | 3 |
| | unsatisfactory | 2 | 2 | 3 | 4 | 4 | 4 |
| Sweetness | Pronounced | 5 | 3 | 4 | 10 | 12 | 9 |
| | Moderate (main | 14 | 13 | 15 | 8 | 5 | 9 |
| | indicator) | | | | | | |
| | Mild | 3 | 4 | 1 | 2 | 3 | 2 |
| Intensity of | Excessively | 2 | 1 | 4 | 6 | 5 | 6 |
| fruit, | pronounced, | | | | | | |
| caramel, or | unpleasant | | | | | | |
| berry flavor | Moderately | 15 | 16 | 13 | 9 | 11 | 10 |
| | pronounced, | | | | | | |
| | pleasant (main | | | | | | |
| | indicator) | | | | | | |
| | Not sufficiently | 3 | 3 | 3 | 5 | 4 | 4 |
| | pronounced | | | | | | |

The data presented indicate that, in terms of organoleptic properties, the claimed food product FoxySpot has a higher percentage of positive ratings than the prototype (Myospot compositions).

The applicant would like to note that Example 2 from the original application materials partially demonstrates an improvement in the quality of life of consumers—the absence of hypersalivation, snoring, and mouth breathing in 85% of consumers, which are factors that impair the quality of life. In addition, Examples 3, 4, 5, and 6 also refer to information that contributes to an improvement in the quality of life of consumers: prevention of caries (reduction of the risk of its occurrence), prevention of oral and throat infections, and prevention of recurrence for consumers who have undergone orthodontic treatment. Thus, the technical result in the form of improving the quality of life of consumers is also achieved through the implementation of the claimed invention—the FoxySpot food product according to paragraph 1 of the refined formula of the invention.

1. A food product for myofunctional prevention of orofacial disorders in human oral cavity, comprising:
biocompatible materials selected from the group consisting of xylitol, agar-agar, carboxymethylcellulose, magnesium stearate, colloidal silicon dioxide,
natural flavoring, and
optionally, natural coloring selected from the group consisting of beet coloring and dry turmeric extract,
wherein the food product following content of components in one tablet in mg: xylitol 120.0, agar-agar 40.0, carboxymethylcellulose 23.0, caramel flavoring 6.0, xanthan gum 5.0, colloidal silicon dioxide 3.0, magnesium stearate 3.0, or xylitol 120.0, agar-agar 40.0, carboxymethylcellulose 20.0, forest berry flavoring 5.0, beetroot coloring 4.0, xanthan gum 5.0, colloidal silicon dioxide 3.0, magnesium stearate 3.0 or xylitol 120.0, agar-agar 40.0, carboxymethylcellulose 23.0, mango flavoring 5.0, dry turmeric extract 1.0, xanthan gum 5.0, colloidal silicon dioxide 3.0, magnesium stearate 3.0.

2. The food product according to claim 1, wherein the food product has a form of a disc with a diameter of 5 to 8 mm and a thickness of 3 mm.

3. The food product according to claim 1, wherein food product has a dissolution time in the oral cavity of 20-60 minutes.

4. A method for treatment of myofunctional prevention of orofacial disorders in the human oral cavity using the food product of claim 1.

5. The method for treatment according to claim 4, wherein the food product has a form of a disc with a diameter of 5 to 8 mm and a thickness of 3 mm.

6. The method for treatment according to claim 4, wherein the food product has a dissolution time in the oral cavity of 20-60 minutes.
